(19) United States
(12) Patent Application Publication
(10) Pub. No.: US 2015/0257684 A1
(43) Pub. Date: Sep. 17, 2015

(54) ELECTRIC PUMP AND DEVICE FOR SUCH PUMP

(71) Applicants: Florian Kandzia, Munchen (DE); Andre Lodwig, Worthsee (DE); Johann Oswald, Grafing (DE)

(72) Inventors: Florian Kandzia, Munchen (DE); Andre Lodwig, Worthsee (DE); Johann Oswald, Grafing (DE)

(21) Appl. No.: 14/203,884

(22) Filed: Mar. 11, 2014

(51) Int. Cl. A61B 5/12

(52) U.S. Cl. CPC A61B 5/126; H04R 2499/10; A61B 2562/168

(57) ABSTRACT

A reversible electric motor driven pump mounted within a housing with one end in communication and seals with the ear canal when inserted therein has a drive shall employing pump controls operably associated with the electric motor to selectively control the start, stop, operating speed, position and direction of the drive shaft in either a clockwise or counterclockwise direction to drive a rotating gear with lever structure configured to reciprocate when driven by the drive shaft to selectively compress the bellows and piston forcing air through a port to create positive pressure is the ear canal in one mode, and expand the bellows and harmonic piston to withdraw air from the ear canal and create negative pressure therein in another mode.

### BACKGROUND OF THE INVENTION

1. Field

This invention relates to hearing testing pumps. In particular, it pertains to a reversible electric motor driven pump with drive shaft employing pump controls operably associated with the electric motor to selectively control the start, stop, operating speed and rotation of the drive shaft in either a clockwise or counterclockwise direction to drive a rotating gear with lever structure configured to reciprocate when driven by the drive, shaft to selectively compress bellows and/or a piston forcing air through a tubing structure to create positive pressure entering art ear canal in one mode, and expand the bellows and/or a piston to withdraw air from the ear canal and create negative pressure therein in another mode.

2. State of the Art

Special hearing tests, namely Tympanometry, employ air pumps to change the pressure in the ear canal during hearing testing. These instruments vary the pressure in the ear canal with a pump, generate a pure tone, and then measure the eardrum responses to the sound tone at different pressures. This produces a series of data measuring how admittance of the reflected sound varies with pressure and is plotted as a tympanogram. Normally, the air pressure in the middle ear canal is the same as ambient pressure, and under normal conditions the air pressure in the middle ear is approximately the same as the ambient pressure since the Eustachian tube opens periodically to ventilate the middle ear to equalize pressure. The tympanograms of a normal person show normal mobility of the eardrum and the conduction hones forming a symmetrical bell shaped curve tympanogram. A skewed tympanogram curve may reveal fluid in the middle ear, perforation of the tympanic membrane, scarring of the tympanic membrane, lack of contact between the condition bones of the middle ear, or a tumor in the middle ear; see http://en.wikipedia.org/wiki/Tympanometry.

One of the key parts of these pressure changing hearing equipment systems is the pump. It should be small, low power, low noise, safe, and inexpensive.

Current tympanometers use linear piston or harmonic/bellow-style pumps. Both concepts typically use a step motor or DC motor driving a threaded bar and a nut to convert the rotational motion into a linear shift. The nut is usually mechanically coupled to a piston or bellows. When activated by the motor, this linear drive alternatively compresses the bellows or piston compressing air or stretches the bellows and retracts the piston to expand from the ear canal as shown in FIGS. 1 and 2. These constructions typically use one or more switches to detect at least one special displacement position, which is needed to calibrate the position of the pump during power-up.

Other pumps use different linear movers, such as belts, tooth belts or levers. These pumps also need a means to detect at least one special displacement position of the mover, typically achieved by one or more switches.

Another pump routinely used for hearing tests is the peristaltic pump, see http://en.wikipedia.org/wiki/Peristaltic pump shown in FIG. 3 and Kusch et al., US 2012/0156074 published Jun. 21, 2012. The peristaltic pump has a flexible tube with an intake fitted inside a circular pump casing. A rotor supporting a number of rollers, shoes, wipers or lobes selectively contacts the tube in a manner to compress the tube as the rotor turns to alternatively compress air, or expand air when operated reversed as the tube opens to its natural state after the passage of the rollers, etc. This pump does not need any position encoding, since it runs continuously.

Consequently, for tympanometer type testing, bellows, pistons, or tube pumps are used. These pumps should have low noise, be bi-directional, lightweight, small low power, safe, provide up to ±600 daPa of pressure. They also should preferably be low cost and easy to service.

Existing pumps have the following drawbacks:

a. Tube pumps age quickly, consume much power, are big and relatively expensive.

b. Bellows and harmonica piston pumps use linear drives, based on threaded control rods or similar constructions. These drives are relatively complex and cannot easily be constructed from off-the-shelf components. The linear drives tend to be bulky and need additional means to calibrate their position.

c. Stepper motors effectively have multiple “toothed” electromagnets arranged around a central gear-shaped piece of iron. The electromagnets are energized by an external control circuit. To make the motor shaft turn, first, one electromagnet is given power, which magnetically attracts the gear's teeth. When the gear's teeth are aligned to the first electromagnet, they are slightly offset from the next electromagnet. So when the next electromagnet is turned on and the first is turned off the gear rotates slightly to align with the next one, and from there the process is repeated. Each of those rotations is called a “step”, with an integer number of steps making a full rotation. In that way, the motor can be turned by a precise angle. The disadvantage of stepper motors is that initial position calibration is needed for step-motor based constructions that use end-switches. Their stepped movement causes these motors tend to generate significant unwanted noise. Moreover, stepper motors need a hold current even when not stepping, thus consuming idle power.

There thus remains a need for an inexpensive, compact, low power, long living pump for tympanometry. The device described below provides such an invention.

### SUMMARY OF THE INVENTION

A hearing pump comprising: a reversible electric motor with a shaft, pump controls operably associated with the motor to selectively control the operating speed and directional drive of the motor shaft in cither a clockwise or counterclockwise rotation. A rotating gear is associated with the shaft with lever structure adapted to reciprocate a bellows or harmonic pump back and forth to alternatively compress or expand air to provide positive or negative pressure in the connected ear canal.

A potentiometer senses the position of the harmonic pump piston or bellows displacement. The controlling circuit uses this signal to initialize the pomp at startup and to know when the maximum displacement is reached.

The potentiometer-based position control allows more sophisticated pump control by the electric motor, and makes the initial position calibration of step-motor based constructions that use end-switches obsolete. The first derivation of the position dpos/dt can also be used as a measure of rotating speed.

The pump can be operated in a wide range of speeds, especially if equipped with a tachometer generator or tachometer-less speed controlling circuit. This allows the pump to reset very quickly.

The pump cannot operate continuously. However, with added valves, it can return to a middle position and continue pumping with relatively short interruption. Typical time constants for off-the-shelf components are 0.5 seconds for such a cycle. It can therefore generate quasi-constant pressure by closing the output terminal with an additional valve, rewind the pump, and then continue pumping. Existing constructions are relatively slow in rewinding, while this new servo-based construction can resume pumping in less than a second.

The invention thus provides a rotationally driven harmonica or piston style pump controlled by an electric motor with a potentiometer sensing the displacement of a gear driven connecting rod or lateral extension lever activating a bellows or piston, which alters the pressure within the ear canal during testing to provide different pressure levels of up to ±600 daPa of pressure within the ear canal.

The linkage from the motor to a bellows may be configured as a rocker arm to provide non-linear compression and expansion of the bellows.

The hearing test pump preferably uses an off-the-shelf (RC-model) servo as driving the gearing and electric motor. For the bellows, a disposable harmonica-style pipette may be used as a replaceable pump element in a configuration allowing a user to periodically replace the same. A potentiometer is used as the position encoder. Such a potentiometer is already Included in commercially available servos used in RC-models. To improve motor startup torque and speed control a tachometer may be employed. Also a negative-impedance-circuit may be included to improve DC-motor startup torque and speed control without the need of a tachometer. Such a negative impedance circuit can compensate the DC resistance of the motor coils and therefore provide a significantly improved speed control.

The complete pump unit thus uses user exchangeable components, and is itself a user-exchangeable module.

The pump preferably is equipped with a vent that allows it to open to the ambiance. This allows the pump to initialize to a defined start position without any pressure being built up.

In one configuration, an additional second valve may be included to allow quasi-constant pressure by adding a quick “rebreathing-cycle” via the bellows or piston. During such a “rebreathing cycle”, the second valve would close the air duct to the ear. A first valve then opens the pump to the ambience. The pump drive returns back to a middle position, then the first valve closes and the second valve opens again, thus resuming operation.

In one embodiment, the pump is structured as a one-piece housing with a sealing ear tip similar to that shown in Heller et al., U.S. Pat. No. 4,688,582 FIG. 1 to FIG. 3. It is held within the ear canal to send and receive signals as the device is held next to the ear. In another embodiment, the pump is structured as a desktop unit placed on a support surface with an ear probe similar to that shown in FIG. 1 in Kiar et al., U.S. Pat. No. 4,002,161 (showing an optional head mount). The pump is connected via an electrical wire and flexible tube associated with the ear probe so that the tube connects to the pump port at one end and the other end of the probe is adapted to fit within and seal the ear canal as signals are sent and received by the desktop housing. In still other variations, the desk top unit with a probe and probe connection is employed. It is structured with the option of connecting to an extender that contains the pump unit, which in turn connects to the ear probe that is designed to fit within and seal the ear canal as signals are sent mid received by the main device and the added pumping unit.

The invention thus provides an inexpensive, compact, low power, long living pump for tympanometry.

### DESCRIPTION OF THE DRAWINGS

FIG. 1 illustrates a conventional tympanometer pump using a threaded rod, driven by a stepper or DC-motor, and a piston pump.

FIG. 2 shows a conventional tympanometer pump, using a threaded rod, driven by a stepper or DC-motor, and a bellows pump.

FIG. 3 illustrates a peristaltic pump.

FIG. 4 is an embodiment of a bellows pump, operated by a gear motor, rudder arm lever and driving rod connected to the bellows.

FIG. 5 shows the embodiment of FIG. 4 with the rudder arm lever directly connected to the bellows.

FIG. 6 illustrates a general tympanometer setup using the invention as its pump.

FIG. 7 illustrates an example of a tachometer-less motor driver circuit with negative output impedance.

FIG. 8 illustrates the general typanometer setup of FIG. 6 with an additional valve providing a breathing cycle.

### DESCRIPTION OF THE ILLUSTRATED EMBODIMENTS

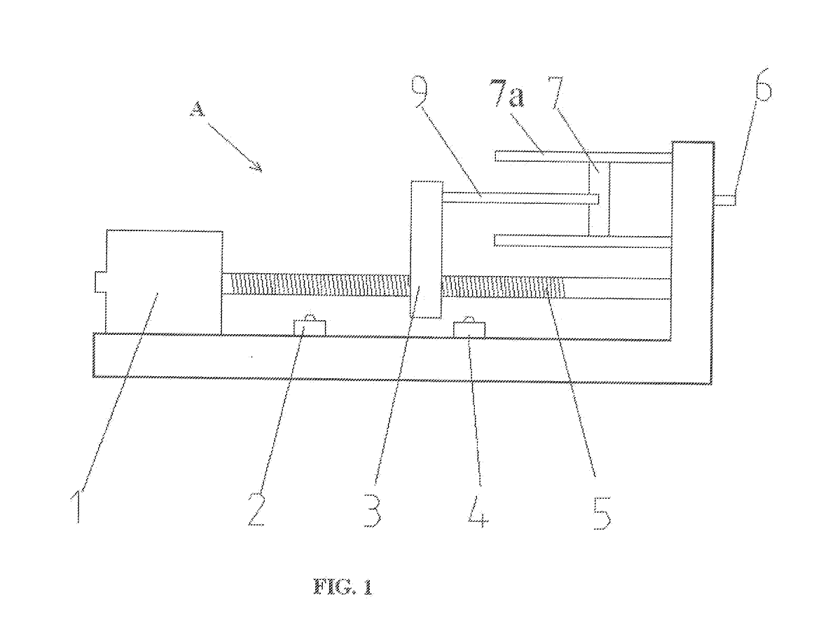

FIG. 1 illustrates a conventional tympanometer pump (A) using a threaded rod 5, driven by a stepper or DC-motor 1, and a piston pump (A). A Step- or DC motor 1 turns a threaded rod 5 that moves a slider 3 back and forth depending upon the rotation of the rod 5. The slider 3 reciprocates the piston rod 9 and piston 7 within a cylinder 7a to provide a compression and decompression cycle forcing air into and oat of a port 6 in communication with the ear canal. End switches 2 and 4 are contacted by the slider 3 to signal the motor 1 to reverse the direction of the threaded rod 5, and are used to calibrate the initial position.

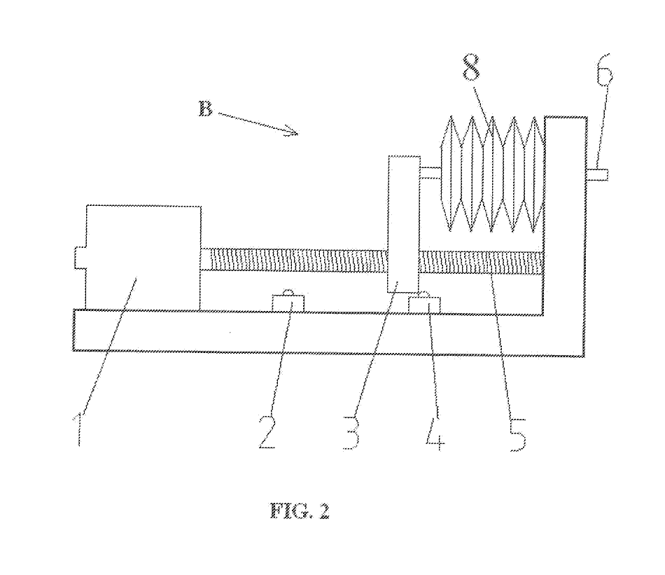

FIG. 2 shows the conventional tympanometer pump (B), using a threaded rod 5, driven by a stepper or DC-motor 1, and a bellows pump (B). A Step- or DC motor 1 turns a threaded rod 5 that moves a slider 3. The slider 3 alternatively compresses and expands the bellows 8 as it reverses position to force air into and out of a port 6 in communication with the ear canal. Again, end switches 2 and 4 are contacted by the slider 3 to signal the motor 1 to reverse the direction of the threaded rod 5, and are used to calibrate the initial position.

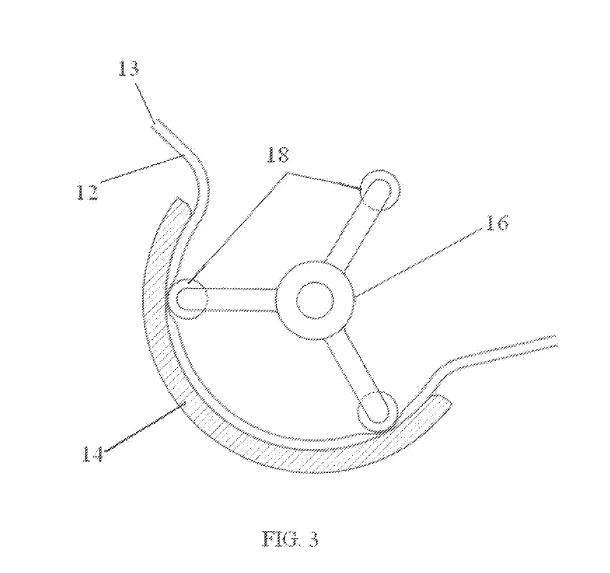

FIG. 3 illustrates a typical peristaltic pump (C) used with current typanometers. It has a flexible tube 12 with an intake 13 fitted inside a circular pump casing 14. A rotor 16 supporting a number of rollers, shoes, wipers, or lobes 18, all referred to hereafter as “rollers”, which selectively contact the tube 12 in a manner to compress the tube 12 as the rotor 16 turns to alternatively force fluids through a nozzle (not shown) by compression, and then draw in fluids as the tube opens to its natural state after the passage of the rollers 18.

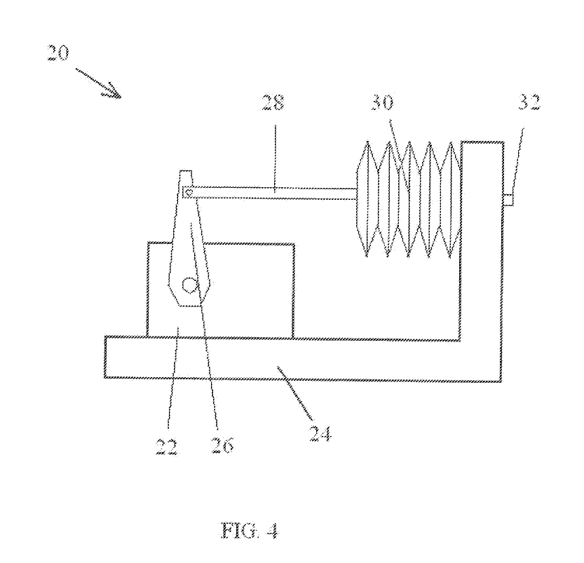

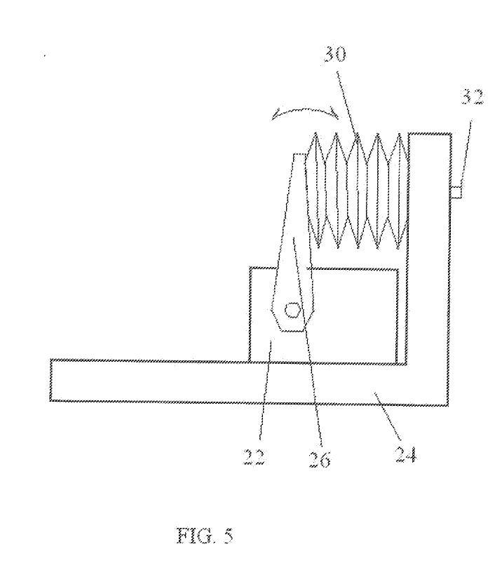

An example of the present invention is shown in FIGS. 4 and 5. The rotationally driven hearing pump 20 comprises a reversible electric gear motor 22 mounted within a housing 24. The gear motor 22 drives a rocker rudder arm 26 with a driving rod 28 operably associated with bellows 30. Compressing the bellows 30 generates positive pressure at its air inlet/outlet 32. Retracting the bellows 30 generates negative pressure at the air inlet/outlet 32. FIG. 5 illustrates an embodiment that does not need a driving rod 28 and makes use of the fact that a bellows 30 does not need to be operated strictly linear.

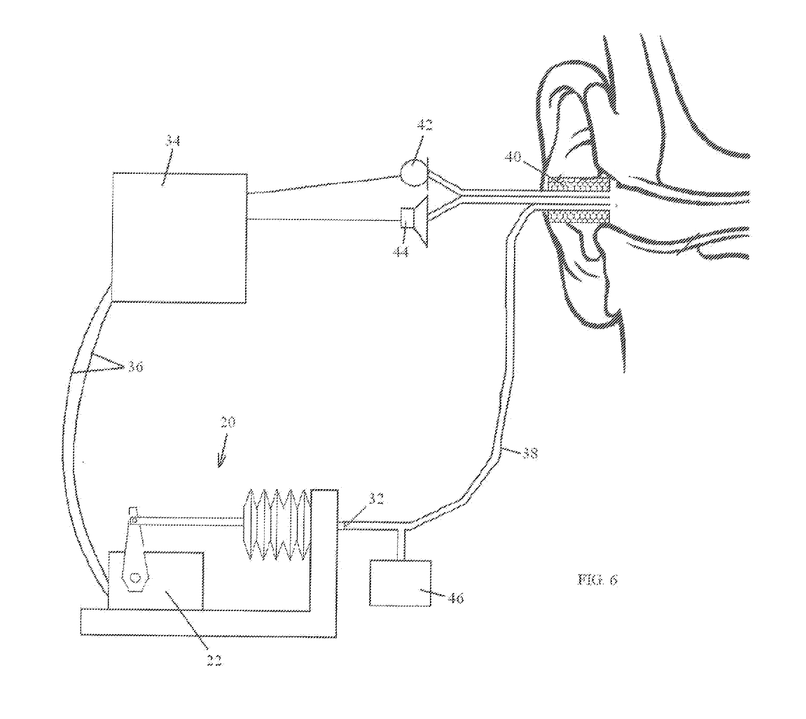

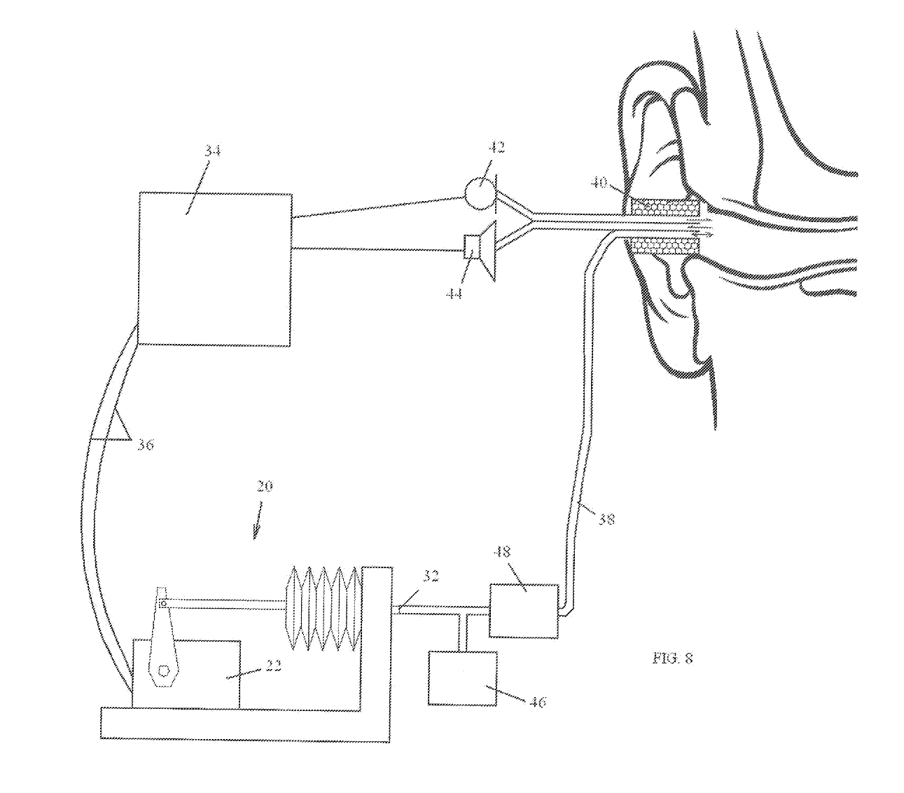

FIG. 6 illustrates a tympanometry setup using the invention. The rotationally driven hearing pump 20 is driven by a controller unit 34 controlling a pump drive consisting of a gear motor 22 driving a rocker rudder arm 26 with a driving rod 28 operably associated with bellows 30 of FIG. 4. The controller unit 34 is connected via connections 36 to the gear motor 22 and potentiometer (not shown) of the gear motor assembly. The pump outlet 32 is connected via a tube 38 to an ear probe 40, which also is associated with a microphone 42 and a loudspeaker 44. A release valve 46 opens the air ducts to the ambience during initialization. An optional valve 48 shown in FIG. 8 would close the tube connection to the ear probe during re-initialization to allow quasi-continuous pumping.

During tympanometry, the loudspeaker 44 would be driven with a tone of typically 226 Hz, and the microphone 42 records the sound pressure of this tone in the ear canal. During the recording, the pump 20 would generate a varying pressure from typically −400 dPa to 400 dPa. The recorded sound pressure as a function of pump-generated air pressure would then be processed and plotted as the typanogram.

Typically, the controller unit 34 would consist of a digital, controller with AD and DA converters, amplifiers, motor drivers, interfaces and display (all not shown).

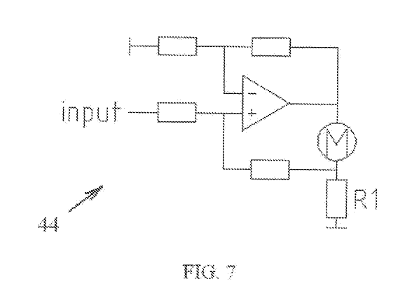

FIG. 7 shows an example circuit 44 to provide the negative impedance driver to the DC motor 22. Alternatively, a tachometer generator (not shown) can be used as feedback providing the motor speed to the driver and/or controller 34. The feedback signal from current sensing resistor R1 is used to compensate the motor's coil resistance this allowing a much more precise response to the input signal of the driver.

FIG. 8 illustrates the general typanometer setup of FIG. 6 with an additional second valve 48 providing a breathing cycle. The second valve 48 is Inserted into the tubing 38 leading to the ear. During the “rebreathing cycle”, the second valve 48 closes the tubing 38 air duct to the ear. The release valve 46 then opens the pump to the ambience to allow quasi-constant pressure by adding a quick “rebreathing-cycle” for the bellows 8 or piston 7. The pump drive returns back to a middle position, then the release valve 46 closes and the second valve 48 opens again, thus resuming operation.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

1. A rotationally driven hearing testing pump comprising:
a) a reversible electric motor with a gear mechanism, providing an output shaft with a lever,
b) a potentiometer operably associated to the gear mechanism, adapted to read the position of the output shaft,
c) a bellows or harmonic piston operably associated with the lever to alternatively vary output by compressing the bellows or harmonic piston to displace air out a port in one mode and expand the bellows or harmonic piston to draw in air through the port in another mode,
d) pump controls operably associated with the reversible electric motor and the potentiometer to selectively control the start, stop, operating speed, position and direction of the shaft rotation in either a clockwise or counterclockwise direction, and
e) output structure associated with the bellows or harmonic piston output in communication with the ear canal to alter the pressure therein in response to the action of the bellows or harmonic piston.

2. A rotationally driven hearing testing pump according to claim 1, wherein the bellows or harmonic piston displacement generates up to +600 daPa of pressure in a compression mode, and up to −600 daPa of pressure in a de-compression mode.

3. A rotationally driven hearing testing pump according to claim 1, including a tympanometer type device structured to seal within the ear canal with
i. a tone generator and at least one loudspeaker to transmit tones into the ear canal, and
ii. at least one microphone to record sound pressure at different pump pressures to produce tympanograms.

4. A rotationally driven hearing testing pump according to claim 1, wherein the reversible electric motor is a servo assembly as used in radio-controlled (RC) models.

5. A rotationally driven hearing testing pump according to claim 1, wherein the bellows comprises a disposable harmonica-style pipette.

6. A rotationally driven hearing testing pump according to claim 1, wherein the harmonic piston has a joint-less connecting rod.

7. A rotationally driven bearing testing pump according to claim 1, wherein the potentiometer is used as a position encoder.

8. A rotationally driven hearing testing pump according to claim 1, wherein the electric motor includes a tachometer for motor startup and speed control.

9. A rotationally driven hearing testing pump according to claim 1, including a negative-impedance-circuit associated with the electric motor for DC-motor startup torque and speed control.

10. A rotationally driven hearing testing pump according to claim 1, including a first valve associated with the bellows or harmonic piston output to open the pump to ambient air, and a second valve that closes the structure in communication with the ear canal, to interrupt pumping by consecutively closing the second valve, opening the first valve, repositioning the lever, closing the first valve and opening the second valve.

11. A rotationally driven hearing testing pump according to claim 1, including a housing encasing the pump motor, potentiometer, bellows or harmonic piston, and pump control components structured to allow access to those pump components constructed of user-exchangeable modules.

12. A rotationally driven hearing testing pump according to claim 11, wherein the housing is structured to be directly held to the ear under test.

13. A rotationally driven bearing testing pump according to claim 3, wherein the output structure comprises an ear probe connected via electric wires and at least one flexible tube structured to seal into the ear canal under test.

14. A rotationally driven hearing testing pump according to claim 13, including an extension unit connecting the ear probe to a main unit housing the pump motor, potentiometer, bellows or harmonic piston, and pump control components for the main unit to do tympanometric tests.
